(19) United States
(12) Patent Application Publication
(10) Pub. No.: US 2024/0269167 A1
(43) Pub. Date: Aug. 15, 2024

(54) A MUCOSAL RE-EPITHELIALIZATION COMPOSITION

(71) Applicant: BIOINTELLIGENT TECHNOLOGY SYSTEMS SLU, Barcelona (ES)

(72) Inventor: Maher Atari Abouasi, Barcelona (ES)

(21) Appl. No.: 18/258,658

(22) PCT Filed: Dec. 20, 2021
(86) PCT No.: PCT/EP2021/086775

§ 371 Date: Jun. 21, 2023

(30) Foreign Application Priority Data
Dec. 21, 2020 (EP) 20383132.6

(51) Int. Cl. A61K 33/14; A61K 9/00; A61K 9/08; A61K 31/192; A61K 31/4425; A61K 33/42; A61P 1/02; A61P 17/02

(52) U.S. Cl. CPC A61K 33/14; A61K 9/0043; A61K 9/006; A61K 9/08; A61K 31/192; A61K 31/4425; A61K 33/42; A61P 1/02; A61P 17/02

(57) ABSTRACT

The use of a composition comprising a therapeutically effective amount of sodium dihydrogen phosphate and sodium chloride as mucosal re-epithelialization promoter, particularly to the oral, nasal, and paranasal mucosal. A composition comprising sodium dihydrogen phosphate, sodium chloride, potassium hydrogen phosphate, potassium chloride, calcium chloride, and a salt of a divalent metal different from calcium, particularly magnesium chloride; processes for their preparation; a kit containing it and its use as mucosal re-epithelialization promoter.

This application claims the benefit of European Patent Application EP20383132.6 filed Dec. 21, 2020.

The present invention relates to the use of a composition comprising a therapeutically effective amount of sodium dihydrogen phosphate and sodium chloride as mucosal re-epithelialization promoter, particularly to the oral, nasal, and paranasal mucosal. It also relates to a composition comprising sodium dihydrogen phosphate, sodium chloride, potassium hydrogen phosphate, potassium chloride, calcium chloride, and a salt of a divalent metal different from calcium, particularly magnesium chloride; processes for their preparation; a kit containing it and its use as mucosal re-epithelialization promoter.

### BACKGROUND ART

The epithelium provides an effective barrier between the airway and subepithelium. This barrier supports epithelial cells, tight junctions, and adherens junctions, which ensure a strong cell-to-cell contact. If epithelial cohesion and integrity are destroyed by surgery, injury, inflammation or infection, the mucosa can be invaded by pathogens and environmental antigens, resulting in the development of mucosal diseases or conditions, and pain.

When there is a injure in the epithelium, the haemostatic phase is initiated. The injured site is sealed rapidly off by the forming blood clot that basically originated from blood coagulation. Extravasated platelets are activated and aggregate together with other blood-derived cells such as neutrophils and red blood cells in the blood clot, also termed blood coagulum. The main component of the extracellular matrix is the newly formed fibrin meshwork that also includes other proteins for cell adhesion such as fibronectin and vitronectin. This conglomerate of cells and the fibrin-rich matrix is frequently termed “provisional extracellular matrix” as it will be later replaced by the granulation tissue. The formation of the blood clot is also the kick-start for the recruiting of inflammatory cells into the injured site. The inflammatory phase parallels the haemostatic phase. Neutrophils are attracted by chemokines, the complement system, and by peptides released during cleavage of fibrinogen. Extravasation and migration of cells in the surrounding tissue is controlled by endothelial cells. Neutrophils and monocytes appear at injured sites within one and 24 h respectively. Neutrophils clean the wound site as they kill invading bacteria and release proteases before they are removed via phagocytosis. In general, resolution of inflammation is a controlled process involving lipid mediators. The primarily catabolic inflammatory process is transient, but crucial for the following steps of the anabolic phase of “new tissue formation”.

Due to the pronounced pain and inflammation around the injured area during its healing, a high hygienic conditions and mucosa care can be challenging for the patients. Therefore, several strategies for accelerate the healing of the injured mucosa have been disclosed in the state of the art. One of these strategies involves the use of antibiotic, antimycotic or antifungal drugs for controlling the (re)infection in the injured area. Another strategy is based on the hydration control of the injured area. In fact, the use of polymers having mucoadherent and film-forming properties, such as polyvinylpyrrolidone and hyaluronic acid product, has been broadly used for reducing water evaporation, which is crucial for accelerate the healing.

In the particular case of oral cavity, the teeth are attached to the bone and to the periodontal tissue (periodontium) through the gingiva, periodontal ligament (PDL), cementum and alveolar bone. Periodontal diseases and conditions usually result in swelling up of the gingiva, bleeding, or formation of periodontal pockets. Such symptoms (ulcers and vesicles) could be due to a single to multiple etiologic agents corresponding to varied diagnoses and treatment regimens. Usually, these symptoms are reversible without causing severe systemic, but sometimes can progress to advanced periodontal diseases, displaying symptoms of dull pain and tooth mobility. Gingival diseases can occur due to microbial attack from the plaque biofilm which is usually bacterial in nature; or also by viral, fungal, and immune-mediated mechanisms. Some systemic conditions also influence the gingiva which allows diagnosing systemic diseases and treating these conditions appropriately. Furthermore, injure and teeth extractions can also cause a ginvival condition that can be aggrieved by additional infections. A broad number of oral care compositions in form of mouthwash, gels and toothpaste are commercially available for the treatment of ginvival diseases and disorders. These compositions usually contain chlorhexidine, triclosan or cetylpiridinium chloride as active ingredients in combination with thymol, and other essential oils as menthol and eucalyptus and a variable amount of alcohol. However, even the use of the above-mentioned oral care compositions, the healing of the mucosa is still slow, lasting about 1 month from the disruption of the epithelium. It means that the risk of (re)infection is maintained during a long period of time causing at the same time pain and discomfort for the patient, which causes in turn an insufficient hygiene and in a high percentage of treatment abandonment. Furthermore, the frequent use of the above-mentioned oral care compositions is associated with some drawbacks, such as teeth sensitivity and mucosa dissolution.

Further, in the particular case of nasal cavity (or nasal fossa) it is a large air-filled space above and behind the nose in the middle of the face. The lower portion of the cavity includes the nasal epithelium, which is an essential part of the respiratory tract. The epithelium of the nasal cavity traps fine particles such as dust, pollen and smoke helping to remove unwanted pathogens and allergens; and warming and moistening the air entering the body before it reaches the lungs, making it easier for the body to absorb. The mucus protects the olfactory epithelium and allows odours to dissolve so that they can be detected by olfactory receptor neurons. Inhaled allergens, including dust, pollen, moulds, animal dander, and grass may react with the mucous membrane; resulting in a drying, irritation and disruption of the epithelium of the nasal cavity. These factors along with dry climates, changing seasons, and pollution can all lead to nasal cavity disease or disorder, which may cause cracking and bleeding of the nose as well as various other acute or chronic conditions. Furthermore, wounds, surgeries and traumatic injuries are other sources of nasal diseases or conditions; which can also by aggrieved by additional infections. Therefore, methods and compositions for moisturizing and establishing the nasal integrity of the nasal mucosa has been disclosed in the state of the art. On one hand, the use of aqueous base multi-dose nasal sprays has been disclosed in the state of the art. However, these compositions generally remain in the nasal cavity only for a short period of time and, therefore, may be relatively ineffective. On the other hand, air humidifiers for the treatment of nasal dryness have been also used. However, humidifiers generally require that the subject utilizing the humidifier remain proximate to the humidified air for a long period of time and such use may therefore be problematic or undesirable in some instances.

And, in the particular case of paranasal cavity, the mucosa of nose continues with the paranasal sinuses. The paranasal sinuses, along with the turbinate, facilitate the function of the nasal space in the warming and humidification of air and contribute to the body's defences against microbial ingress. In particular, the lining of the sinuses (ciliated columnar epithelium) produces mucus, which is moved by the action of cilia in a synchronised pattern around the sinus often against gravity, and in the case of the frontal sinus not by the most direct route, to the ostia where drainage occurs into the nasal space. From the nasal space the mucus passes into the nasopharynx and is swallowed. However, in the presence of a disease it is the interruption of this basic process, usually by reduced ciliary activity or obstruction, that causes symptoms. The ostia of the anterior ethmoid, frontal and maxillary sinuses are closely approximated in the middle meatus, such that inflammation related to middle meatal soft tissue will often involve more than one sinus. The most common paranasal diseases are the inflammatory paranasal sinus disease, causing pain within about one week and can be complicated by an upper respiratory tract infection; the acute rhinosinal disease causing facial pain, nasal congestion, and discharge; and the chronic rhinosinal diseases that are nasal congestion or discharge that persists for eight to 12 weeks. The causes of these diseases are varied, but the most common is the viral origin, regardless chronic rhinosinus disease which usually have bacterial origin rather than viral. Once the diagnosis has been made the aim is the selection of the best treatment. However, treatment of acute sinusitis is based on relief of symptoms and does not involve antibiotics unless the patient is pyrexial or there is evidence of spread of infection beyond the confines of the sinus. It means that the duration of the illness is usually not influenced (reduced) by the symptomatic treatment which can last up to four weeks. Commonly, the treatment is based on topical nasal decongestants and saline irrigation of the nasal cavity. Topical decongestants such as ephedrine or xylometazoline constrict the nasal lining, widening the paranasal sinus ostia, facilitating drainage by ciliary activity. Most decongestants are provided in form of a spray delivery device and are easily administered; but on the contrary, droplet preparations require more careful administration to be effective. Unfortunately, excessive decongestant usage will cause localized nasal discomfort. Further, nasal decongestants should not be used for more than 7 days because of rebound mucosal swelling when the medication is stopped. Saline irrigation of the nasal cavity is the equivalent of a warm salty mouthwash in that it shifts surface debris and will facilitate sinus drainage. In practice this is delivered using a 10 ml or 20 ml syringe with the patient vigorously washing out their nasal cavity while leaning over a sink. Further, when infection is spreading beyond the confines of the paranal sinuses or the patient is pyrexial, with a pussy nasal discharge, an appropriate antibiotic is needed (i.e., amoxicillin, cephalosporin, doxycycline, or clarithromycin). However, if a patient is clinically unwell, or shows signs of orbital involvement, urgent referral to a hospital setting is recommended. On the other hand, treatment of chronic disease is the same as for acute disease, with nasal irrigation and nasal decongestants in the first instance. Nasal decongestants may be used for a prolonged period on the basis that their use is restricted to once daily. When polyps are present, steroids, either topical or systemic, may be prescribed.

Chronic paranasal disease or recurrent paranasal acute disease does not respond to conventional medical therapy may require surgery. This can involve treating a deviated nasal septum, removing polyps, removing, or trimming turbinates, or increasing the size of the ostium from the maxillary sinus and removal of excess tissue in the middle meatus.

In conclusion, it has been disclosed in the state of the art several treatments for restoring the mucosal epithelium, particularly to the oral, nasal, and paranasal mucosa. However, most of these treatments are based on minimising the symptoms associate to the disease, but not treating the origin, which does not reduce the duration of the disease. It means that the disease is maintained for more than 1 month causing pain and discomfort, with a high risk of (re)infection or systemic complications.

Thus, from what is known in the state of the art there is still the need to develop an effective composition for the mucosal re-epithelialization without having the disadvantages mentioned above.

### SUMMARY OF INVENTION

The inventors have surprisingly found that a composition comprising a therapeutically effective amount of sodium dihydrogen phosphate and sodium chloride is useful for promoting mucosal re-epithelialization; particularly the oral, nasal, or paranasal mucosa. In particular, the composition of the present invention increases and accelerates the re-epithelization and protect the epithelium of the mucosa. Furthermore, it also reduces pain, inflammation, water loss, wound and ulcer healing allowing an improvement of the general comfort of the patient during the process of mucosa healing. Besides, the composition of the invention also minimizes the risk of (re)infections avoiding systemics complications, such as respiratory diseases or sepsis; as well as the percentage of subsequent interventions by forming a protective layer over the epithelial to prevent the adhesion of external agents such as bacteria, virus, and fungi.

Therefore, as it is shown in the experimental section, the compositions of the present invention are useful for the treatment of oral mucosal diseases or conditions (such as gingivitis, even those associated with periodontal diseases and dental extractions), nasal mucosal diseases and conditions (such as acute and chronic rhinitis and nasal dryness) and paranasal mucosal diseases and conditions (such as chronic and acute inflammatory paranasal sinus disease: chronic sinusitis, paranasal dryness, rhinitis, rhinolith, Killian or anthrocoanal polyposis, mucocele, deforming polyposis or woakes syndrome, retention cysts and Schneiderian papilloma).

In the particular case of the oral mucosa, the inventors have also found that the compositions of the present invention which are capable of promoting re-epithelialization of the oral mucosa are also useful for avoiding implant failure by promoting the engraftment of the implant with the surrounding gingiva, specifically when dental implants are used.

The inventors have also found that a composition comprising sodium dihydrogen phosphate in a concentration from 8 to 12 mM in the solution; sodium chloride in a concentration from 130 to 140 mM in the solution; potassium hydrogen phosphate in a concentration from 1.4 to 2.2 mM in the solution; potassium chloride in a concentration from 2.3 to 3.1 mM in the solution; a calcium salt; particularly calcium chloride, in a concentration from 0.7 to 2 mM in the solution; and a salt of a divalent metal different from calcium, particularly magnesium chloride, in a concentration from 0.2 to 1.5 mM in the solution; is specially advantageous for use in promoting the mucosal re-epithelization, that is for use as mucosal re-epithelization promoter. It is specially advantageous for the re-epithelization of the oral, nasal, or paranasal mucosa.

In particular, these compositions having an low amount of divalent metal salts; particularly an amount of a calcium salt (particularly calcium chloride) from 0.7 to 2 mM in the solution and a salt of a divalent metal different from (particularly magnesium chloride), in a concentration from 0.2 to 1.5 mM in the solution; allows an even faster re-epithelization of the mucosa and a higher reduction of pain either when the composition is administered directly or by way of any means containing the composition. For example, for the oral mucosa, the composition of the first and the second aspect of the invention allows a faster re-epithelization of the mucosa and a higher reduction of pain, as well as a faster recovery and closure of the gingival wound was observed even by its direct application or by submerging the suture with the composition before using it to close the wound. As it is shown in the experimental section, the oral mucosa is restored in about 15 days instead of about one month of the commercially available comparative composition.

Finally, due to the acceleration of the mucosa healing, a reduction of the time of treatment is required. This fact, in combination with a reduction of patient discomfort (caused by a low pain sensation and inflammation of the injured area) results in a higher compliance of the treatment, and a low percentage of abandonment.

Thus, the first aspect of the invention refers to a water-based salt solution composition comprising: a therapeutically effective amount of sodium dihydrogen phosphate; a therapeutically effective amount of sodium chloride; optionally, one or more pharmaceutically acceptable excipients of carriers; and optionally, one or more additional active ingredients; for use in promoting mucosal re-epithelialization, that is as mucosal re-epithelialization promoter.

The second aspect of the invention refers to a composition comprising: sodium dihydrogen phosphate in a concentration from 8 to 12 mM in the solution; sodium chloride in a concentration from 130 to 140 mM in the solution; potassium hydrogen phosphate in a concentration from 1.4 to 2.2 mM in the solution; potassium chloride in a concentration from 2.3 to 3.1 mM in the solution; calcium salt, particularly calcium chloride, in a concentration from 0.7 to 2 mM in the solution; a salt of a divalent metal different from calcium, particularly magnesium chloride, in a concentration from higher than 0.2 to 1.5 mM in the solution; optionally, one or more pharmaceutically acceptable excipients of carriers; and optionally, one or more additional active ingredients.

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly throughout the specification and claims unless an otherwise expressly set out definition provides a broader definition.

For the purposes of the present invention, any ranges given include both the lower and the upper end-points of the range. Ranges given, such as temperatures, times, weights, and the like, should be considered approximate, unless specifically stated.

The terms “percentage (%) by weight”, “weight/weight %” and “w/w %” have the same meaning and are used interchangeable. They refer to the percentage of each ingredient of the composition in relation to the total weight of the composition.

The concentration of the solutes in the solution composition of the invention are expressed in molar concentration. The terms “molar concentration”, “molarity”, “amount concentration” and “substance concentration” have the same meaning and they are used interchangeable. The molarity is a measure of the concentration of a chemical species, in particular of a solute in a solution, in terms of amount of the substance per unit volume of solution. In chemistry, the most commonly used unit for molarity is the number of moles per litter, having the unit symbol mol/L or mol·dm−3 in SI unit. A solution with a concentration of 1 mol/L is said to be 1 molar (it means that: 1 mol/m3=10−3 mol/dm3=10−3 mol/L=10−3 M=1 mmol/L=1 mM.).

Molarity is most commonly expressed in units of moles of solute per litre of solution. For use in broader applications, it is defined as amount of substance of solute per unit volume of solution, or per unit volume available to the species, represented by lowercase c:

$$c = \frac{n}{V} = \frac{N}{{N}_{A} V} = \frac{C}{{N}_{A}} .$$

Here, n is the amount of the solute in moles, N is the number of constituent particles present in volume V (in litres) of the solution, and NA is the Avogadro constant, since 20 May 2019 defined as exactly 6.02214076×1023 mol−1. The ratio NN is the number density C.

As it is mentioned above, the composition of the first aspect of the invention promotes the mucosal re-epithelialization; particularly the mucosa of the oral and nasal cavity (such as the gingiva en the oral cavity). Thus, the first aspect of the invention refers to a composition for use in promoting mucosal re-epithelialization; it means for use as mucosa re-epithelialization promoter.

The term “mucosal re-epithelialization” has the meaning generally understood in the state of the art; that is promoting the restoration of the epithelium by development and formation of epithelium to re-stablish the structural and functional integrity of the mucosa. The term “mucosa” refers to a layer formed by epithelium and the underlying loose connective tissue, that lines the inner walls of organs that are in contact with the outside of the body. It is usually associated with numerous mucus secreting glands. In general, they have protective, secretory, and absorptive functions, and house highly developed and specialized immune subsystems.

In an embodiment, the mucosa is selected from the group consisting of oral mucosa, nasal mucosa, and paranasal mucosa. In an embodiment, the mucosa is the oral mucosa of the oral cavity. For the purpose of the present invention, the oral mucosa encompasses the gingiva. In an embodiment, the mucosa is the nasal mucosa of the nasal cavity. In an embodiment, the mucosa is the paranasal mucosa of the paranasal cavity.

In an embodiment, the re-epithelialization of the mucosa comprises wound healing; ulcer healing; healing of lesions caused by bacteria, virus, and fungi; healing of bacterial, viral, or fungal infection and forming a protective layer over the epithelium to prevent the adhesion of external agents such as bacteria, virus, and fungi. In an embodiment, the composition of the first aspect of the invention is useful for the treatment of a mucosal disease or condition which involves the re-epithelialization of the mucosa. In an embodiment, the composition of the first aspect of the invention is useful for the treatment of the oral mucosal disease or condition which involves the re-epithelialization of the oral mucosa. Examples of oral mucosal diseases or conditions which involves the re-epithelialization of the oral mucosa include, but not limited to, gingiva diseases or conditions; dental implant replacement and dental extraction Examples of gingiva diseases or conditions includes, but without limitation, gingivitis and/or periodontitis including those cases after: dental extractions, dental implant replacement and any oral or maxillofacial surgery; candidiasis; ulcers; sores and stomatitis, In an embodiment, the composition of the first aspect of the invention is useful for the treatment of the nasal mucosal disease or condition which involves the re-epithelialization of the nasal mucosa. Examples of nasal disease or condition includes, but without limitation, acute and chronic rhinitis, and nasal dryness. In an embodiment, the composition of the first aspect of the invention is useful for the treatment of the paranasal mucosal disease or condition which involves the re-epithelialization of the nasal mucosa. Examples of paranasal disease or condition includes, but without limitation, acute and chronic sinusitis, paranasal dryness, rhinitis, rhinolith, Kilian or anthrocoanal polyposis, mucocele, deforming polyposis or woakes syndrome, retention cysts, and Schneiderian papilloma

In an embodiment, the composition for use of the first aspect of the invention comprises contacting once or twice a day the mucosa with the composition as defined in the first aspect of the invention. In an embodiment, the composition for use of the first aspect of the invention comprises contacting once a day the mucosa with the composition. Commonly, it is performed in the morning or in the evening after eating and usually immediately after cleaning and brushing teeth. In an embodiment, the composition for use of the first aspect of the invention comprises contacting twice a day the mucosa with the composition. Commonly, it is performed in the morning and in the evening after eating and usually immediately after cleaning and brushing teeth. It is also recommended not to eat after the use of the composition of the present invention; particularly from 15 to 30 min after the use of the composition of the present invention.

In an embodiment, when the composition for use of the first aspect of the invention is an oral composition, then the contact with the oral mucosa is performed by rinsing, washing, cleaning. Commonly, the contact of the oral mucosa and the composition is performed from 1 min to 3 min; particularly about 2 min. In an embodiment, when the composition for use of the first aspect of the invention is a nasal or paranasal composition, then the contact of the mucosa is performed by spraying, droppings by the patient or cleaning in the same surgical act in nasal or paranasal by the professional. Commonly, the contact of the nasal or paranasal mucosa and the composition is performed from 1 min to 5 min; particularly about 2 min.

In an embodiment, the use of the composition is performed from 4 days to 10 days. In an embodiment, the use of the composition is performed from 5 days to 9 days. In an embodiment, the use of the composition is performed from 6 days to 8 days. In an embodiment, the use of the composition is performed for about 7 days.

In an embodiment, the composition for use of the first aspect of the invention, which comprises contacting once or twice a day the mucosa with the composition is performed for 4 to 10 days. In an embodiment, the composition for use of the first aspect of the invention, which comprises contacting twice a day the mucosa with the composition for 7 days. In an embodiment, the oral composition for use of the first aspect of the invention comprises contacting once a day the oral mucosa with the composition for 1 min to 3 min during 4 days to 10 days. In an embodiment, the oral composition for use of the first aspect of the invention comprises contacting once a day the oral mucosa with the composition for about 2 min for 7 days. In an embodiment, the oral composition for use of the first aspect of the invention comprises contacting twice a day the oral mucosa with the composition for 1 min to 3 min during 4 days to 10 days. In an embodiment, the oral composition for use of the first aspect of the invention comprises contacting twice a day the oral mucosa with the composition for about 2 min for 7 days.

The composition for use of the first aspect of the invention comprises a therapeutically effective amount of each one of the ingredients. The expression “therapeutically effective amount” as used herein, refers to the amount of the active ingredients that, when administered, which is sufficient to promote mucosal re-epithelialization. The particular dose of the active ingredients administered according to this invention will of course be determined by the skilled in the art regarding the particular circumstances surrounding the case, including the particular condition being treated, and the similar considerations.

The composition for use of the first aspect of the invention comprises a therapeutically effective amount of sodium dihydrogen phosphate (i.e., NaH₂PO₄) and sodium chloride (i.e., NaCl). In an embodiment, the composition for use comprises sodium dihydrogen phosphate is in a concentration from 8 to 140 mM in the solution and sodium chloride is in a concentration from 8 to 140 mM in the solution. In an embodiment, the composition for use comprises sodium dihydrogen phosphate is in a concentration from 8 to 12 mM in the solution and sodium chloride is in a concentration from 130 to 140 mM in the solution. In an embodiment, the composition for use comprises sodium dihydrogen phosphate is in a concentration from 130 to 140 mM in the solution and sodium chloride is in a concentration from 8 to 12 mM in the solution.

In an embodiment, the composition for use further comprises a therapeutically effective amount of potassium hydrogen phosphate (i.e., KHPO₄) and a therapeutically effective amount of potassium chloride (i.e., KCl). In an embodiment, the composition for use further comprises potassium hydrogen phosphate in a concentration from 1.4 to 2.2 mM in the solution, and potassium chloride in a concentration from 2.3 to 3.1 mM in the solution.

In an embodiment, the composition for use comprises:
a therapeutically effective amount of sodium dihydrogen phosphate;
a therapeutically effective amount of sodium chloride;
a therapeutically effective amount of potassium hydrogen phosphate;
a therapeutically effective amount of potassium chloride;
optionally, one or more pharmaceutically acceptable excipients of carriers; and
optionally, one or more additional active ingredients.

In an embodiment, the composition for use comprises:
sodium dihydrogen phosphate in a concentration from 8 to 12 mM in the solution;
sodium chloride in a concentration from 130 to 140 mM in the solution;
potassium hydrogen phosphate in a concentration from 1.4 to 2.2 mM in the solution;
potassium chloride in a concentration from 2.3 to 3.1 mM in the solution;
optionally, one or more pharmaceutically acceptable excipients of carriers; and
optionally, one or more additional active ingredients.

In an embodiment, the composition for use comprises:
sodium dihydrogen phosphate in a concentration from 130 to 140 mM in the solution;
sodium chloride in a concentration from 8 to 12 mM in the solution;
potassium hydrogen phosphate in a concentration from 1.4 to 2.2 mM in the solution;
potassium chloride in a concentration from 2.3 to 3.1 mM in the solution;
optionally, one or more pharmaceutically acceptable excipients of carriers; and
optionally, one or more additional active ingredients.

In an embodiment, the composition for use further comprises a therapeutically effective amount of calcium salt and a therapeutically effective amount of salt of a divalent metal different from calcium. For the purpose of the invention, the calcium salt is formed by calcium divalent cation (Ca2+) and an anion. In an embodiment, the anion of the calcium salt is chloride, then, the calcium salt is calcium chloride (i.e., CaCl₂). For the purpose of the present invention the salt of a divalent metal different from calcium is formed by a divalent metal cation different from calcium and an anion. In an embodiment, the divalent metal different from calcium is magnesium. In an embodiment, the anion of the salt of the divalent metal different from calcium is chloride. In an embodiment, the salt of a divalent metal different from calcium is magnesium chloride (i.e., MgCl₂).

In an embodiment, the composition for use further comprises a calcium salt which is calcium chloride (i.e., CaCl₂) and a salt of a divalent metal different from calcium which is magnesium chloride (i.e., MgCl₂). In an embodiment, the composition for use further comprises a calcium salt; particularly calcium chloride, in a concentration from 0.7 to 15 mM in the solution and a salt of a divalent metal different from calcium; particularly magnesium chloride, in a concentration from 0.2 to 12 mM in the solution.

In an embodiment, the composition for use further comprises a calcium salt; particularly calcium chloride, in a concentration from 0.7 to 12 mM in the solution. In an embodiment, the composition for use further comprises a calcium salt; particularly calcium chloride, in a concentration from 0.7 to 7 mM in the solution. In an embodiment, the composition for use further comprises a calcium salt; particularly calcium chloride, in a concentration from 0.7 to 4 mM in the solution. In an embodiment, the composition for use further comprises a calcium salt; particularly calcium chloride, in a concentration from 0.7 to 2 mM in the solution.

In an embodiment, the composition for use further comprises a salt of a divalent metal different from calcium; particularly magnesium chloride, is in a concentration from 2 to 9 mM in the solution. In an embodiment, the composition for use further comprises a salt of a divalent metal different from calcium; particularly magnesium chloride, is in a concentration from 2 to 5 mM in the solution. In an embodiment, the composition for use further comprises a salt of a divalent metal different from calcium; particularly magnesium chloride, is in a concentration from 2 to 2 mM in the solution. In an embodiment, the composition for use further comprises a salt of a divalent metal different from calcium; particularly magnesium chloride, is in a concentration from 2 to 1.5 mM in the solution.

In an embodiment, the composition for use further comprises a calcium salt; particularly calcium chloride, in a concentration from 0.7 to 12 mM in the solution; a salt of a divalent metal different from calcium; particularly magnesium chloride, is in a concentration from 2 to 9 mM in the solution. In an embodiment, the composition for use further comprises a calcium salt; particularly calcium chloride, in a concentration from 0.7 to 7 mM in the solution; a salt of a divalent metal different from calcium; particularly magnesium chloride, is in a concentration from 2 to 5 mM in the solution. In an embodiment, the composition for use further comprises a calcium salt; particularly calcium chloride, in a concentration from 0.7 to 4 mM in the solution; a salt of a divalent metal different from calcium; particularly magnesium chloride, is in a concentration from 2 to 2 mM in the solution. In an embodiment, the composition for use further comprises a calcium salt; particularly calcium chloride, in a concentration from 0.7 to 2 mM in the solution; a salt of a divalent metal different from calcium; particularly magnesium chloride, is in a concentration from 2 to 1.5 mM in the solution.

In an embodiment, the composition for use comprises:
a therapeutically effective amount of sodium dihydrogen phosphate;
a therapeutically effective amount of sodium chloride;
a therapeutically effective amount of potassium hydrogen phosphate;
a therapeutically effective amount of potassium chloride;
a therapeutically effective amount of calcium salt; particularly calcium chloride;
a therapeutically effective amount of salt of a divalent metal different from calcium; particularly magnesium chloride;
optionally, one or more pharmaceutically acceptable excipients of carriers; and
optionally, one or more additional active ingredients.

In an embodiment, the composition for use comprises:
a therapeutically effective amount of sodium dihydrogen phosphate;
a therapeutically effective amount of sodium chloride;
a therapeutically effective amount of potassium hydrogen phosphate;
a therapeutically effective amount of potassium chloride;
a therapeutically effective amount of calcium chloride;
a therapeutically effective amount of magnesium chloride;
optionally, one or more pharmaceutically acceptable excipients of carriers; and
optionally, one or more additional active ingredients.

In an embodiment, the composition for use comprises:
sodium dihydrogen phosphate in a concentration from 8 to 12 mM in the solution;
sodium chloride in a concentration from 130 to 140 mM in the solution;
potassium hydrogen phosphate in a concentration from 1.4 to 2.2 mM in the solution;
potassium chloride in a concentration from 2.3 to 3.1 mM in the solution;
a calcium salt in a concentration from 0.7 to 15 mM in the solution;
a salt of a divalent metal different from calcium in a concentration from 0.2 to 12 mM in the solution
optionally, one or more pharmaceutically acceptable excipients of carriers; and
optionally, one or more additional active ingredients.

In an embodiment, the composition for use comprises:
sodium dihydrogen phosphate in a concentration from 130 to 140 mM in the solution;
sodium chloride in a concentration from 8 to 12 mM in the solution;
potassium hydrogen phosphate in a concentration from 1.4 to 2.2 mM in the solution;
potassium chloride in a concentration from 2.3 to 3.1 mM in the solution;
a calcium salt in a concentration from 0.7 to 15 mM in the solution;
a salt of a divalent metal different from calcium in a concentration from 0.2 to 12 mM in the solution
optionally, one or more pharmaceutically acceptable excipients of carriers; and
optionally, one or more additional active ingredients.

In an embodiment, the composition for use comprises:
sodium dihydrogen phosphate in a concentration from 8 to 12 mM in the solution;
sodium chloride in a concentration from 130 to 140 mM in the solution;
potassium hydrogen phosphate in a concentration from 1.4 to 2.2 mM in the solution;
potassium chloride in a concentration from 2.3 to 3.1 mM in the solution;
calcium chloride in a concentration from 0.7 to 15 mM in the solution;
magnesium chloride in a concentration from 0.2 to 12 mM in the solution;
optionally, one or more pharmaceutically acceptable excipients of carriers; and
optionally, one or more additional active ingredients.

In an embodiment, the composition for use comprises:
sodium dihydrogen phosphate in a concentration from 130 to 140 mM in the solution;
sodium chloride in a concentration from 8 to 12 mM in the solution;
potassium hydrogen phosphate in a concentration from 1.4 to 2.2 mM in the solution;
potassium chloride in a concentration from 2.3 to 3.1 mM in the solution;
calcium chloride in a concentration from 0.7 to 15 mM in the solution;
magnesium chloride in a concentration from 0.2 to 12 mM in the solution;
optionally, one or more pharmaceutically acceptable excipients of carriers; and
optionally, one or more additional active ingredients.

In an embodiment, the composition for use comprises:
sodium dihydrogen phosphate in a concentration from 8 to 12 mM in the solution;
sodium chloride in a concentration from 130 to 140 mM in the solution;
potassium hydrogen phosphate in a concentration from 1.4 to 2.2 mM in the solution;
potassium chloride in a concentration from 2.3 to 3.1 mM in the solution;
a calcium salt in a concentration from 0.7 to 12 mM in the solution;
a salt of a divalent metal different from calcium in a concentration from 2 to 9 mM in the solution;
optionally, one or more pharmaceutically acceptable excipients of carriers; and
optionally, one or more additional active ingredients.

In an embodiment, the composition for use comprises:
sodium dihydrogen phosphate in a concentration from 130 to 140 mM in the solution;
sodium chloride in a concentration from 8 to 12 mM in the solution;
potassium hydrogen phosphate in a concentration from 1.4 to 2.2 mM in the solution;
potassium chloride in a concentration from 2.3 to 3.1 mM in the solution;
a calcium salt in a concentration from 0.7 to 12 mM in the solution;
a salt of a divalent metal different from calcium in a concentration from 2 to 9 mM in the solution;
optionally, one or more pharmaceutically acceptable excipients of carriers; and
optionally, one or more additional active ingredients.

In an embodiment, the composition for use comprises:
sodium dihydrogen phosphate in a concentration from 8 to 12 mM in the solution;
sodium chloride in a concentration from 130 to 140 mM in the solution;
potassium hydrogen phosphate in a concentration from 1.4 to 2.2 mM in the solution;
potassium chloride in a concentration from 2.3 to 3.1 mM in the solution;
calcium chloride in a concentration from 0.7 to 12 mM in the solution;
magnesium chloride in a concentration from 2 to 9 mM in the solution;
optionally, one or more pharmaceutically acceptable excipients of carriers; and
optionally, one or more additional active ingredients.

In an embodiment, the composition for use comprises:
sodium dihydrogen phosphate in a concentration from 130 to 140 mM in the solution;
sodium chloride in a concentration from 8 to 12 mM in the solution;
potassium hydrogen phosphate in a concentration from 1.4 to 2.2 mM in the solution;
potassium chloride in a concentration from 2.3 to 3.1 mM in the solution;
calcium chloride in a concentration from 0.7 to 12 mM in the solution;
magnesium chloride in a concentration from 2 to 9 mM in the solution;
optionally, one or more pharmaceutically acceptable excipients of carriers; and
optionally, one or more additional active ingredients.

In an embodiment, the composition for use comprises:
sodium dihydrogen phosphate in a concentration from 8 to 12 mM in the solution;
sodium chloride in a concentration from 130 to 140 mM in the solution;
potassium hydrogen phosphate in a concentration from 1.4 to 2.2 mM in the solution;
potassium chloride in a concentration from 2.3 to 3.1 mM in the solution;
calcium chloride in a concentration from 0.7 to 12 mM in the solution;
magnesium chloride in a concentration from 4 to 9 mM in the solution;
optionally, one or more pharmaceutically acceptable excipients of carriers; and
optionally, one or more additional active ingredients.

In an embodiment, the composition for use comprises:
sodium dihydrogen phosphate in a concentration from 130 to 140 mM in the solution;
sodium chloride in a concentration from 8 to 12 mM in the solution;
potassium hydrogen phosphate in a concentration from 1.4 to 2.2 mM in the solution;
potassium chloride in a concentration from 2.3 to 3.1 mM in the solution;
calcium chloride in a concentration from 0.7 to 12 mM in the solution;
magnesium chloride in a concentration from 4 to 9 mM in the solution;
optionally, one or more pharmaceutically acceptable excipients of carriers; and
optionally, one or more additional active ingredients.

In an embodiment, the composition for use comprises:
sodium dihydrogen phosphate in a concentration from 8 to 12 mM in the solution;
sodium chloride in a concentration from 130 to 140 mM in the solution;
potassium hydrogen phosphate in a concentration from 1.4 to 2.2 mM in the solution;
potassium chloride in a concentration from 2.3 to 3.1 mM in the solution;
a calcium salt in a concentration from 0.7 to 7 mM in the solution;
a salt of a divalent metal different from calcium in a concentration from 2 to 5 mM in the solution;
optionally, one or more pharmaceutically acceptable excipients of carriers; and
optionally, one or more additional active ingredients.

In an embodiment, the composition for use comprises:
sodium dihydrogen phosphate in a concentration from 130 to 140 mM in the solution;
sodium chloride in a concentration from 8 to 12 mM in the solution;
potassium hydrogen phosphate in a concentration from 1.4 to 2.2 mM in the solution;
potassium chloride in a concentration from 2.3 to 3.1 mM in the solution;
a calcium salt in a concentration from 0.7 to 7 mM in the solution;
a salt of a divalent metal different from calcium in a concentration from 2 to 5 mM in the solution;
optionally, one or more pharmaceutically acceptable excipients of carriers; and
optionally, one or more additional active ingredients.

In an embodiment, the composition for use comprises:
sodium dihydrogen phosphate in a concentration from 8 to 12 mM in the solution;
sodium chloride in a concentration from 130 to 140 mM in the solution;
potassium hydrogen phosphate in a concentration from 1.4 to 2.2 mM in the solution;
potassium chloride in a concentration from 2.3 to 3.1 mM in the solution;
a calcium salt in a concentration from 0.7 to 7 mM in the solution;
magnesium chloride in a concentration from 2 to 5 mM in the solution;
optionally, one or more pharmaceutically acceptable excipients of carriers; and
optionally, one or more additional active ingredients.

In an embodiment, the composition for use comprises:
sodium dihydrogen phosphate in a concentration from 130 to 140 mM in the solution;
sodium chloride in a concentration from 8 to 12 mM in the solution;
potassium hydrogen phosphate in a concentration from 1.4 to 2.2 mM in the solution;
potassium chloride in a concentration from 2.3 to 3.1 mM in the solution;
a calcium salt in a concentration from 0.7 to 7 mM in the solution;
magnesium chloride in a concentration from 2 to 5 mM in the solution;
optionally, one or more pharmaceutically acceptable excipients of carriers; and
optionally, one or more additional active ingredients.

In an embodiment, the composition for use comprises:
sodium dihydrogen phosphate in a concentration from 8 to 12 mM in the solution;
sodium chloride in a concentration from 130 to 140 mM in the solution;
potassium hydrogen phosphate in a concentration from 1.4 to 2.2 mM in the solution;
potassium chloride in a concentration from 2.3 to 3.1 mM in the solution;
a calcium salt in a concentration from 0.7 to 4 mM in the solution;
a salt of a divalent metal different from calcium in a concentration from 0.2 to 2 mM in the solution
optionally, one or more pharmaceutically acceptable excipients of carriers; and
optionally, one or more additional active ingredients.

In an embodiment, the composition for use comprises:
sodium dihydrogen phosphate in a concentration from 8 to 12 mM in the solution;
sodium chloride in a concentration from 130 to 140 mM in the solution;
potassium hydrogen phosphate in a concentration from 1.4 to 2.2 mM in the solution;
potassium chloride in a concentration from 2.3 to 3.1 mM in the solution;
a calcium salt in a concentration from 0.7 to 4 mM in the solution;
a salt of a divalent metal different from calcium in a concentration from 0.2 to 2 mM in the solution
optionally, one or more pharmaceutically acceptable excipients of carriers; and
optionally, one or more additional active ingredients.

In an embodiment, the composition for use comprises:
sodium dihydrogen phosphate in a concentration from 130 to 140 mM in the solution;
sodium chloride in a concentration from 8 to 12 mM in the solution;
potassium hydrogen phosphate in a concentration from 1.4 to 2.2 mM in the solution;
potassium chloride in a concentration from 2.3 to 3.1 mM in the solution;
a calcium salt in a concentration from 0.7 to 4 mM in the solution;
magnesium chloride in a concentration from 0.2 to 2 mM in the solution;
optionally, one or more pharmaceutically acceptable excipients of carriers; and
optionally, one or more additional active ingredients.

In an embodiment, the composition for use comprises:
sodium dihydrogen phosphate in a concentration from 8 to 12 mM in the solution;
sodium chloride in a concentration from 130 to 140 mM in the solution;
potassium hydrogen phosphate in a concentration from 1.4 to 2.2 mM in the solution;
potassium chloride in a concentration from 2.3 to 3.1 mM in the solution;
a calcium salt in a concentration from 0.7 to 2 mM in the solution;
a salt of a divalent metal different from calcium in a concentration from 0.2 to 1.5 mM in the solution;
optionally, one or more pharmaceutically acceptable excipients of carriers; and
optionally, one or more additional active ingredients.

In an embodiment, the composition for use comprises:
sodium dihydrogen phosphate in a concentration from 130 to 140 mM in the solution;
sodium chloride in a concentration from 8 to 12 mM in the solution;
potassium hydrogen phosphate in a concentration from 1.4 to 2.2 mM in the solution;
potassium chloride in a concentration from 2.3 to 3.1 mM in the solution;
a calcium salt in a concentration from 0.7 to 2 mM in the solution;
a salt of a divalent metal different from calcium in a concentration from 0.2 to 1.5 mM in the solution;
optionally, one or more pharmaceutically acceptable excipients of carriers; and
optionally, one or more additional active ingredients.

In an embodiment, the composition for use comprises:
sodium dihydrogen phosphate in a concentration from 8 to 12 mM in the solution;
sodium chloride in a concentration from 130 to 140 mM in the solution;
potassium hydrogen phosphate in a concentration from 1.4 to 2.2 mM in the solution;
potassium chloride in a concentration from 2.3 to 3.1 mM in the solution;
a calcium salt in a concentration from 0.7 to 2 mM in the solution;
magnesium chloride in a concentration from 0.2 to 1.5 mM in the solution;
optionally, one or more pharmaceutically acceptable excipients of carriers; and
optionally, one or more additional active ingredients.

In an embodiment, the composition for use comprises:
sodium dihydrogen phosphate in a concentration from 130 to 140 mM in the solution
sodium chloride in a concentration from 8 to 12 mM in the solution;
potassium hydrogen phosphate in a concentration from 1.4 to 2.2 mM in the solution;
potassium chloride in a concentration from 2.3 to 3.1 mM in the solution;
a calcium salt in a concentration from 0.7 to 2 mM in the solution;
magnesium chloride in a concentration from 0.2 to 1.5 mM in the solution;
optionally, one or more pharmaceutically acceptable excipients of carriers; and
optionally, one or more additional active ingredients.

In an embodiment, the composition for use comprises:
10 mM of sodium dihydrogen phosphate;
137 mM of sodium chloride;
1.8 mM of potassium hydrogen phosphate;
2.7 mM of potassium chloride;
10.8 mM of calcium chloride;
8.4 mM of magnesium chloride;
optionally, one or more pharmaceutically acceptable excipients of carriers; and
optionally, one or more additional active ingredients.

In an embodiment, the composition for use comprises:
8 mM of sodium dihydrogen phosphate;
138 mM of sodium chloride;
1.4 mM of potassium hydrogen phosphate;
2.5 mM of potassium chloride;
0.9 mM of calcium chloride;
0.5 mM of magnesium chloride;
optionally, one or more pharmaceutically acceptable excipients of carriers; and
optionally, one or more additional active ingredients.

In an embodiment, the composition for use comprises:
138 mM of sodium dihydrogen phosphate;
8 mM of sodium chloride;
1.4 mM of potassium hydrogen phosphate;
2.5 mM of potassium chloride;
0.9 mM of calcium chloride;
0.5 mM of magnesium chloride;
optionally, one or more pharmaceutically acceptable excipients of carriers; and
optionally, one or more additional active ingredients.

In an embodiment, the composition for use of the first aspect of the invention has a pH from 6.8 to 7.4. In an embodiment, the composition for use of the first aspect of the invention has a pH about 7. For the purpose of the present invention, the pH is measured by a pH meter. The use of the compositions as defined above in the first aspect of the invention having a pH from 6.8 to 7.4 are especially advantageous because the optimal pH for the appropriate functionality of the progenitor cells of any tissue. Therefore, these compositions allow a faster re-epithelization of the mucosa and a higher reduction of pain; in the particular case of the oral mucosa, a faster recovery and closure of the gingival wound was observed.

The term “about” or “around” as used herein refers to a range of values ±10% of a specified value. For example, the expression “about 7” includes ±10% of 7, i.e., from 6.3 to 7.7. The pH can be measured by any method disclosed in the state of the art.

The composition for use of the first aspect of the invention, optionally comprises one or more pharmaceutically acceptable excipients or carriers. In an embodiment, composition for use of the first aspect of the invention, comprises one or more pharmaceutically acceptable excipients or carriers.

The term “pharmaceutically acceptable” refers to any composition, compound, or material suitable for use in the pharmaceutical technology. For the purpose of the present invention, the term “pharmaceutically acceptable excipients or carriers” refers to that excipients or carriers for preparing compositions with medical use. The appropriate excipients and/or carriers, and their amounts, can readily be determined by those skilled in the art according to the type of formulation being prepared. Examples of appropriate excipients or carriers includes surfactant, humectant, preservative agent, sweetener, flavouring, colorant, pH-regulating agent, gelling agent, thickener, emollient, opacifying agent, chelating agent, tonicity adjusting agent, solvent, co-solvent, and diluent.

The term “surfactant” refers to a material which lowers the surface tension of a liquid and the interfacial tension between two liquids, allowing their easier spreading. Surfactants have a hydrophilic head that is attracted to water molecules and a hydrophobic tail that repels water and simultaneously attaches itself to oil and grease in dirt. These opposing forces loosen the dirt and suspend it in the water, having the ability to remove it from surfaces such as the human skin, textiles, and other solids, when surfactants are dissolved in water.

The term “binging agent” or “binder” or “thickening agent” or “thickener” or “viscosity agent” have the same meaning and are herein used interchangeably. They refer to a material that control or modify the rheology in terms of viscosity and thixotropy without substantially modifying its other properties.

The term “gelling agent” refers to a material which forms a gel by the action of an aqueous medium, such as water or an aqueous solution of an organic acid (such as aqueous citric or acetic acid), a base (such as sodium bicarbonate or sodium tetraborate solution) or alcohol (e.g. an aqueous lower alkanol such as aqueous ethanol or isopropanol).

The term “humectant” refers to a material that hold or retain moisture.

The term “emollient” agent refers to a material that softens and soothes the mucosa cavity in order to correct dryness and scaling of the cavity, lubricating the mucosal surface, and encouraging tissue water retention.

The term “preservative” refers to a material that prevents or reduces or slows down microbial growth, providing that the stability of the solution is not affected.

The term “sweetener” refers to a component that presents a sweet taste.

The terms “colorant” or “pigment” have the same meaning and are herein used interchangeably. They refer to a coloured substance that, when suspended in a liquid medium, becomes paint.

The term “pH-regulating” agent refers to acids or bases that can be used to adjust the pH of the finished product to the desired level, without affecting the stability of the solution and maintaining a pleasant feel for the patient.

The term “opacifying agent” refers to a material which, when added to a formulation having a transmittance of from about 55% to 100% when measured at 440 nm wavelength, is capable of producing a formulation having a transmittance reading of about 20% or less when measured at a 440 nm wavelength.

The term “chelating agent” refers to any compound capable of coordinating or binding metal ions (commonly found in natural water such as calcium, magnesium, iron, and manganese metal ions) forming water soluble metal complexes

The term “tonicity adjusting agent” refers to any compound that alters the osmolality of an aqueous solution. Typically, tonicity agents are used to adjust the osmolality of a solution to bring it closer to the osmotic pressure of body fluids, such as blood or plasma.

The term “solvent” refers to any compound having the ability to increase the solubility of a solute in a solution. For the purpose of the present invention, as the composition is a water-based salt solution, the solvent is a “miscible organic solvent”. The term “miscible organic solvent” refers to an organic solvent that, when combined, form a single phase, which means that the mixture thus obtained is “monophasic” under specified conditions of component concentrations and temperature among others. Further, the term “water-miscible organic solvent” refers to an organic solvent that can form a monophasic solution with water at the temperature at which the reaction is carried out. As used herein, the term “monophasic” refers to a reaction medium that includes only one liquid phase, and also a method employing such a reaction medium. Some examples of monophasic mediums are water, aqueous solutions, and solutions containing aqueous and organic solvents that are miscible with each other. Examples include ethanol, ethylamine, formic acid, glycerol, triethylene glycol, pyridine, acetone and acetonitrile.

The term “co-solvent” refers to a solvent that is used in combination with a primary solvent to increase the solubility of the solutes in the solution. For the purpose of the present invention, as the composition is a water-based salt solution, the co-solvent is also a “miscible organic solvent” as defined above.

The term “diluent” refers to a nontoxic pharmacologically acceptable carrier which, when mixed with the active ingredient, renders it suitable for producing the composition of the invention. The diluent is sufficiently compatible with the active ingredient as to permit adequate concentrations of the latter.

As it is mentioned above, the mucosa can be the oral mucosa, the nasal mucosa, and the paranasal mucosa.

In an embodiment, when the composition is for use in the oral mucosa, then the composition is selected from the group consisting of mouthwash, mouth rinse, oral solution, dental gel, dispersion through a water jet, breath freshener, or gargle. In an embodiment, when the composition is for use in the oral mucosa, then the composition is a mouthwash or a mouth rinse. The appropriate excipients and/or carriers, and their amounts, can readily be determined by those skilled in the art taking into account the oral administration of the composition being prepared. These compositions can be prepared according to methods well known in the state of the art.

In an embodiment, when the composition is for use in the nasal or paranasal mucosa, then the composition is selected from the group consisting of nasal non-pressurized dispenser or nasal pressurized dispenser. In an embodiment, when the composition is for use in the nasal or paranasal mucosa, then the composition is a non-pressurized disperser; particularly selected from the group consisting of nasal sprays and nasal drops. In an embodiment, when the composition is for use in the nasal or paranasal mucosa, then the composition is a pressurized disperser; particularly in form of a unit dose, bi-dose or multi-dose devices comprise at least one propellant agent and wherein the composition as defined in the present invention is either dissolved. Examples of appropriate pressurized dispersers for the present invention includes nasal aerosol, nasal nebulizer, and nasal insufflator. The propellant is the agent that supplies the necessary pressure within the aerosol, nebulizer, or insufflator system to expel the composition of the invention from the container. Propellants are commonly classified as liquefied or compressed gases having vapour pressures generally exceeding atmospheric pressure. Examples of suitable propellants for the present invention includes hydrocarbons, especially halogenated derivatives of methane, ethane, and propane, low molecular weight hydrocarbons such as the butanes and pentanes, and compressed gases such as carbon dioxide, nitrogen, and nitrous oxide. Mixtures of propellants are frequently used to obtain desirable pressure, delivery, and spray characteristics. These nasal or paranasal compositions of the present invention can be prepared according to methods well known in the state of the art.

The composition for use of the first aspect of the invention, optionally comprises one or more additional active ingredients. In an embodiment, the composition for use of the first aspect of the invention, comprises one or more additional active ingredients. Examples of appropriate additional active agents include biocide agents such as antibiotics, antivirals and antifungal agents (chlorhexidine or cetylpiridinium chloride); antinflammatory agents, vitamins, growing factors and nutrients, hydrating agents such as film-forming agents (polyvinylpyrrolidone, hyaluronic acid and derivatives thereof); additional mucosa regenerating agents (allantoïne, panthenol, and tocopheryl acetate); disinfectant agents (peracetic acid, ethyl alcohol, isopropyl alcohol, quaternary ammonium containing compounds, chlorhexidine, phenols, glutaraldehyde, iodophors, ortho-phthalaldehyde and hydrogen peroxide). When the composition is for use in the oral mucosa, then the composition can further comprise antiplaque agents (such as triclosan, xylitol, sodium fluoride, and zinc chloride) and polishing agents as additional active ingredients. When the composition is for use in the nasal or paranasal mucosa, then the composition can further comprise antinflammatory agents, vitamins, growing actors and nutrients as additional active ingredients. Examples of appropriate further active ingredients include nitrodioxanes, chlorobutanol, polyhexamethylenebiguanide chlorides, dehydroacetic acid, formaldehyde, isothiazolinones, xylenols, oxazolidines, salicylic acid, phenylmercuries, glutaraldehyde, undecylenic derivatives nitro propanediols, chlorhexidine, azoniadamantanes, imidazolidinylureas, hydantoins, phenoxyethanol, parabens, dichlorobenzyl alcohols, potassium sorbate, sodium benzoate and pyridinetiol oxide.

In an embodiment, the composition for use of the present invention, further comprises one or more biocide agents; particularly bactericides such as cetyl pyridinium. In an embodiment, the composition for use of the present invention further comprises one or more biocidal agents; particularly bactericides such as cetyl pyridinium, in a concentration from 0.1 to 0.5 mM in the solution. In an embodiment, the composition for use of the present invention, further comprises one or more nitrodioxanes, chlorobutanol, polyhexamethylenebiguanide chlorides, dehydroacetic acid, formaldehyde, isothiazolinones, xylenols, oxazolidines, salicylic acid, phenylmercuries, glutaraldehyde, undecylenic derivatives nitro propanediols, chlorhexidine, azoniadamantanes, imidazolidinylureas, hydantoins, phenoxyethanol, parabens, dichlorobenzyl alcohols, potassium sorbate, sodium benzoate and pyridinetiol oxide such as sodium benzoate. In an embodiment, the composition for use of the present invention, further comprises one or more preservatives such as sodium benzoate in a concentration from 1 to 2 mM in the solution. In an embodiment, the composition for use of the present invention, further comprises: one or more biocidal agents; particularly bactericides such as cetyl pyridinium; and one or more antivirals such as sodium benzoate. In an embodiment, the composition for use of the present invention, further comprises: one or more biocidal agents; particularly bactericides, such as cetyl pyridinium, in a concentration from 0.1 to 0.5 mM in the solution; and one or more preservatives or antivirals, such as sodium benzoate, in a concentration from 1 to 2 MM in the solution.

The second aspect of the invention relates to a composition comprising:
sodium dihydrogen phosphate in a concentration from 8 to 12 mM in the solution;
sodium chloride in a concentration from 130 to 140 mM in the solution;
potassium hydrogen phosphate in a concentration from 1.4 to 2.2 mM in the solution;
potassium chloride in a concentration from 2.3 to 3.1 mM in the solution;
calcium salt in a concentration from 0.7 to 2 mM in the solution;
a salt of a divalent metal different from calcium in a concentration from higher than 0.2 to 1.5 mM in the solution;
optionally, one or more pharmaceutically acceptable excipients of carriers; and
optionally, one or more additional active ingredients.

In an embodiment, the composition of the second aspect of the invention comprises:
sodium dihydrogen phosphate in a concentration from 8 to 12 mM in the solution;
sodium chloride in a concentration from 130 to 140 mM in the solution;
potassium hydrogen phosphate in a concentration from 1.4 to 2.2 mM in the solution;
potassium chloride in a concentration from 2.3 to 3.1 mM in the solution;
calcium chloride, in a concentration from 0.7 to 2 mM in the solution;
magnesium chloride, in a concentration from higher than 0.2 to 1.5 mM in the solution;
optionally, one or more pharmaceutically acceptable excipients of carriers; and
optionally, one or more additional active ingredients.

In an embodiment, the composition of the second aspect of the invention comprises:
8 mM of sodium dihydrogen phosphate;
138 mM of sodium chloride;
1.4 mM of potassium hydrogen phosphate;
2.5 mM of potassium chloride;
0.9 mM of calcium chloride;
0.5 mM of magnesium chloride;
optionally, one or more pharmaceutically acceptable excipients of carriers; and
optionally, one or more additional active ingredients.

In an embodiment, the composition of the second aspect of the invention has a pH from 6.8 to 7.4. In an embodiment, the composition for use of the first aspect of the invention has a pH about 7.

These compositions of the second aspect of the invention comprises a low amount of divalent salts, particularly an amount of a calcium salt (particularly calcium chloride) from 0.7 to 2 mM in the solution; and a salt of a divalent metal different from calcium, particularly magnesium chloride, from 0.2 to 1.5 mM in the solution. As it is shown in the experimental section, these compositions allow having a higher re-epithelialization promoting effect over the mucosa and a higher reduction of pain and inflammation. All the embodiments mentioned above for the composition of the first aspect of the invention also applies for the second aspect of the invention.

It is also part of the invention the use of the composition of the second aspect of the invention as promoter of mucosal re-epithelialization; that is a composition of the second aspect of the invention for use in promoting mucosal re-epithelialization. All the embodiments mentioned above for the re-epithelialization promoting effect of the first aspect of the invention also apply herein for the composition of the second aspect of the invention.

It is also a part of the invention a kit comprising a composition of the second aspect of the invention and means to apply the composition to the area to be treated. In an embodiment; said means are explained hereinafter. For the purpose of the invention, the composition is a water-based salt solution composition appropriate for being ready-to-use. In an embodiment, the kit comprising a water-based salt solution composition as defined above and means to apply the composition to the area to be treated, wherein the means to apply the composition are selected from the group consisting of a syringe, a cotton rod, and a textile gauze. However, the composition can be in form of solid form because the specialist (professional) has at his disposal the other elements to prepare the water-based salt solution composition and to apply it to the patient (i.e. means for application). In an embodiment, the composition is in form of a solid composition and the means for preparing the water-based salt solution composition. In this case, the means include a syringe equipped with a disposable perforating needle that is used for drawing water from its container and putting into the container with the composition in solid form.

In an embodiment, when the composition is an oral composition, the means are those means appropriate for applying the composition to the oral cavity. Example of means to apply the composition in the oral cavity include, but not limited to, stitches, gauzes, membranes, sponges (such as collagen sponges or haemostatic sponges), membranes (such as tissue protecting membranes) and biomaterials (such as regenerative biomaterials). In an embodiment, the means are those means appropriate for applying the nasal composition to the nasal cavity. Example of means to apply the composition in the nasal cavity include, but not limited to, stitches, gauzes, membranes, sponges (such as collagen sponges or haemostatic sponges), membranes (such as tissue protecting membranes) and biomaterials (such as regenerative biomaterials). In an embodiment, the means are those means appropriate for applying the composition to the paranasal cavity. Example of means to apply the composition in the paranasal cavity include, but not limited to, stitches, gauzes, membranes, sponges (such as collagen sponges or haemostatic sponges), membranes (such as tissue protecting membranes) and biomaterials (such as regenerative biomaterials).

The composition disclosed in the present application can be prepared following the processes commonly disclosed in the state of the art. In fact, the skilled in the art can determined the preparation process according to the type of formulation being prepared. For the purpose of the present invention, the process for the preparation of the compositions of the first and the second aspect of the invention comprises:
i) Dissolving form 8.0-12 mM of sodium dihydrogen phosphate in 800 ml of water until its total dissolution;
ii) Dissolving from 130-140 mM of sodium chloride in the solution until its total dissolution;
iii) if present, dissolving from 1.4-2.2 mM of potassium hydrogen phosphate in the solution until its total dissolution;
iv) if present, dissolving from 2.3-3.1 mM of potassium chloride in the solution until its total dissolution:
v) if present, dissolving from 0.7-2 mM of calcium salt (particularly chloride) in the solution until its total dissolution;
vi) if present, dissolving from 0.20-1.5 mM of divalent metal different from calcium (particularly magnesium chloride) in the solution until its total dissolution,
vii) optionally, adding one or more pharmaceutically acceptable excipients of carriers; and viii) optionally, adding one or more additional active ingredients;
ix) optionally, adjusting the final volume of the solution to 1000 ml; and
x) optionally, adjusting the pH from 6.8 to 7.4;
being the dissolution of all the components at a temperature from 30 to 37° C., particularly about 37° C.

In an embodiment, the process for the preparation of the composition in solid form comprises drying the water-based solution composition of the first or the second aspect of the invention. The solid form composition is reconstituted into the water-based solution before its use by adding the appropriate amount of water for having the concentration of all the components of the composition as defined herein.

As it is mentioned above, the composition of the first and the second aspect of the invention are appropriate for being used as mucosal re-epithelialization promoter. This aspect could be also formulated as the use of the composition as defined above for the preparation of a medicament for the treatment of a mucosal disease or condition implying a loss of the integrity of the epithelium; particularly of the oral, nasal, or paranasal mucosal cavity. It also relates to a method for the treatment of a mammal suffering or is susceptible to suffer from a mucosal disease or condition implying a loss of the integrity of the epithelium; particularly of the oral, nasal, or paranasal mucosal cavity, the method comprises administering to said mammal an effective amount of the water-based salt solution composition of the present invention.

The use of the composition can be performed by directly applying the composition of the first and the second aspect of the invention to the area to be treated, or alternatively applying the compositions to suitable means as defined above such as stitches, gauzes, membranes, sponges (such as collagen sponges or haemostatic sponges), membranes (such as tissue protecting membranes) and biomaterials (such as regenerative biomaterials) for their subsequent application to the area to be treated.

Therefore, it is also part of the invention a process for re-epithelization which comprises:
a) directly applying the composition of the first and the second aspect of the invention to the area to be treated; or alternatively,
b) applying the compositions to suitable means for subsequent application to those means to the area to be treated.

In an embodiment, the suitable means are selected from those disclosed above, such as for example stitches, gauzes, membranes, sponges (such as collagen sponges or haemostatic sponges), membranes (such as tissue protecting membranes) and biomaterials (such as regenerative biomaterials).

In an embodiment, the area to be treated is selected from the group consisting of mucosa, skin, scalp, connective tissue, and collagen. In an embodiment, the area to be treated is mucosa; particularly selected from oral, nasal, and paranasal.

In an embodiment, the re-epithelization comprises wound healing; ulcer healing; healing of lesions caused by bacteria, virus, and fungi; healing of bacterial, viral, or fungal infection and forming a protective layer over the epithelium to prevent the adhesion of external agents such as bacteria, virus, and fungi. In an embodiment, the re-epithelization comprises wound healing in the area to be treated.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples and drawings are provided by way of illustration, and they are not intended to be limiting of the present invention. Reference signs related to drawings and placed in parentheses in a claim, are solely for attempting to increase the intelligibility of the claim and shall not be construed as limiting the scope of the claim. Furthermore, the present invention covers all possible combinations of particular and preferred embodiments described herein.

### Examples

### 1. Compositions

### 1.1. Compositions of the Invention

The components of the mouthwash of the oral compositions of the invention (Examples 1 and 2) and the spray of the nasal and paranasal compositions (Example 3) are shown in Table 1, wherein the molar concentration of the components is expressed in mM in the water solution.

TABLE 1

| | Mouthwash | | Nasal and |
|---|---|---|---|
| | oral compositions | | paranasal spray |
| Ingredients | Example 1 | Example 2 | Example 3 |
| Sodium chloride (NaCl), | 10 | 8 | 138 |
| Sodium dihydrogen | 137 | 138 | 8 |
| phosphate (NaH₂PO₄), | | | |
| Potassium chloride (KCl), | 2.7 | 2.5 | 2.5 |
| Potassium phosphate | 1.8 | 1.4 | 1.4 |
| (KH₂PO₄), | | | |
| Calcium chloride (CaCl₂), | 10.8 | 0.9 | 0.9 |
| Magnesium chloride (MgCl₂), | 8.4 | 0.50 | 0.50 |
| Cetylpyridium chloride | — | 0.15 | 0.15 |
| Benzoate sodium | — | 1.7 | 1.7 |
| pH | 7.6 | 7 | 7 |

The compositions of the invention of Examples 1-3 as defined above were prepared following the preparation process as disclosed below using the amount of the ingredients specified in Table 1.

### Preparation of the Mouthwash Composition of Examples 1 of the Present Invention:

8 g of NaCl, 0.2 g of KCl, 1.44 g of NaH2PO4, 0.24 g of KH2PO4, 0.80 g of MgCl2, and 1.20 g of CaCl2 were dissolved in 800 ml of water. After all the components were correctly dissolved the volume of the solution was brought to 1 l.

### Preparation of the Mouthwash Composition of Examples 1 of the Present Invention:

8 g of NaCl, 0.2 g of KCl, 2,160 g of NaH2PO4, 0.2 g of KH2PO4, 0.1 g of MgCl2, 0.1 g of CaCl2, 0.05 g of Cetylpyridium chloride and 0.25 g of sodium benzoate were dissolved in 800 ml of water. After all the components were correctly dissolved the volume of the solution was brought to 1 l.

### Preparation of the Spray Composition of Example 3 of the Present Invention:

8 g of NaCl, 0.2 g of KCl, 2,160 g of NaH2PO4, 0.2 g of KH2PO4, 0.1 g of MgCl2, 0.1 g of CaCl2, 0.05 g of Cetylpyridium chloride and 0.25 g of sodium benzoate were dissolved in 800 ml of water. After all the components were correctly dissolved the volume of the solution was brought to 1 l.

### 1.2. Comparative Composition

The comparative composition falling outside the scope of the present invention is a mouthwash composition commercially available with the tradename PERIO.AID by DENTAID. The components of the comparative mouthwash (comparative Example 1) are shown in Table 2, wherein the amount of the components is expressed in weight percent in relation to the total weight of the composition (% by weight).

TABLE 2

| | Comparative Mouthwash |
|---|---|
| | PERIO.AID ® (Comparative |
| Ingredients | Example 1) |
| Cetylpiridinium Chloride | 0.05% |
| Excipients q.s.p. | 1% |
| Water | 80% |
| C.I.42090 | |
| Chlorhexidine Digluconato | 0.12% |

### 2. Oral Study Design

The study was designed as a one week randomized, prospective, double-blinded pilot clinical trial. Ethic approval was obtained from the local committee. This prospective study included patients who required two trans alveolar surgical extractions of inferior third molar or any simple or surgical tooth.

Dental extractions were firstly performed, the administration of the treatments was done and the clinical evolution of the patients was monitored. The data obtained from said monitorization were analysed to determine clinical changes during treatment and after a period of one-week follow-up for clinical parameters.

### 2.1. Population

Patients (female or male) who voluntarily signed the informed consent were enrolled to the study according to the following inclusion and exclusion criteria:

### Inclusion Criteria

Patient ≥14 years old and able to understand the nature of the proposed surgery and to sign the informed consent.
Patients systemically healthy.
Non-smokers patients.
Patients with a healthy periodontium.
No local or systemic antibiotic or antiseptic treatments in the previous 3 months.
No systemic diseases or conditions that could interfere with the study outcomes (pregnant or lactating females, uncontrolled periodontal disease, previous or current history of bisphosphonate treatment, immune deficiencies, uncontrolled diabetes, rheumatoid disease, radiotherapy, chemotherapy, infectious diseases . . . )
Full mouth plaque and bleeding scores <20%.
Patients infected with COVID-19 (positive PCR TEST)

### Exclusion Criteria

Smoker patients
Local or systemic conditions that would interfere with routine periodontal therapy.
Patients taking medications that cause gingival enlargement or the presence of gingival idiopathic overgrowth.
   Removal of Patients from Therapy or Assessment

Participants were free to withdraw from the study at any time, without any prejudice and without having to justify the reason. If the patient discontinued prematurely from the study, any relevant evaluations and observations and reasons for study discontinuation were recorded in the Case Report Form (CRF). The specialist might discontinue a participant from the study if there was a reasonable cause. In case of medical reason for withdrawal, the patient was to be monitored until complete recovery.

### 2.2. Treatments

### 2.2.1. Treatments Administered

The study consisted of two parallel arms in which patients were treated with the composition of the present invention (Examples 1 or 2) or with the comparative composition containing 0.12% Chlorhexidine (Comparative Example 1). Both products were presented in white boxes to make the study double-blind. The compositions were administered twice a day for 7 days after teeth brushing. No eating or drinking was allowed during 1 hour after treatment.

### 2.2.2. Groups:

Control Group (CG): receiving comparative composition of comparative Example 1 (i.e. 0.12% Chlorhexidine mouthwash), and
Test Group (TG): receiving the mouthwash composition of Example 1 or 2 of the invention.

### 2.2.3. Visit Schedule

The study was scheduled in two visits. The first visit was the surgery day (visit 1) and the second which was one week later the visit 1 (visit 2). Table 3 shows the assessments and indicates with an “X” the visits at which they were performed.

TABLE 3

| | Visit 1 | |
|---|---|---|
| | (baseline) | Visit 2 |
| | (surgery day) | (Week 1) |
| Informed consent | X | |
| Collection of clinical data | X | X |
| Oral examination | X | X* |
| Inclusion/exclusion criteria | X | |
| Treatment | X | X |
| Patients dairy: clinical | X | X* |
| hilling measurements, eating | | |
| difficulty and pain in a 0-10 | | |
| scale (VAS) | | |
| Tolerability and post-treatment | 0 | 0 |
| side effects | | |

*Exploratory endpoint.

All clinical measurements were performed by two specialists previously calibrated. All patients taking part in the study received a diagnostic workup including a clinical examination, photographs, and standardized periapical radiographs to evaluate the proposed surgical sites. Clinical registrations were made by the two trained and calibrated specialists.

### 2.2.4. Study Steps

### Presurgical Procedure

Before the surgical procedure, all patients underwent periodontal therapy and received extensive oral hygiene instructions to provide a better oral environment. Full mouth plaque scores (FMPS) (cf. O'Leary T J et al. “The plaque control record”. J. Periodontol. 1972, vol 43(1), pp. 38.) and full mouth bleeding scores (FMBS) (cf. Ainamo J et al. “Problems and proposals for recording gingivitis and plaque”. I. Int. Dent. J., 1975, vol. 25(4), pp. 229-35.) were recorded after the hygienic phase of the periodontal therapy.

Before the surgical procedure, Oral hygiene has been performed on all patients by ultrasound or manual curettage. No surgery was performed until patients reached a full-mouth plaque score (FMPS)<20% and a full-mouth bleeding score (FMBS)<20%. The term full-mouth plaque score (FMPS) refers to the number of stained teeth respect to the number of non-stained teeth. The FMPS can be calculated by any method disclosed in the state of the art. For the purpose of the present invention, the FMPS is calculated by specialists. The term full mouth bleeding score (FMBS) refers to the number of bleed points respect to the number of non-bleeds points The FMBS can be calculated by any method disclosed in the state of the art. For the purpose of the present invention, the FMBS is calculated by specialists.

### Surgical Extraction

The surgical extractions of teeth were carried out with local anaesthetic, raising a mucoperiosteal flap with osteotomy. Extraction of molars, central and/or premolar were performed.

No periodontal dressing was applied postoperatively.

### Postsurgical Procedures

All patients received amoxicillin 500 mg every 8 hr for 7 days. In cases of allergy to amoxicillin, doxycycline 100 mg once a day for 5 days was prescribed. Furthermore, ibuprofen 600 mg every 8 hr for 4 days was also prescribed.

All patients for control group were instructed to rinse with 15 ml of the comparative composition (comparative Example 1 having 0.12% chlorhexidine digluconate) for 1 min twice daily after their regular homecare for 1 week. All patients for test group were ordered to rinse with 15 ml of the test composition of the present invention (Example 1 or 2) for 1 min twice a day after regular homecare for 1 week.

The use of ice packs was recommended for at least 3 hours. All patients were instructed to discontinue tooth brushing at the surgical sites for 7 days. After one month, a professional prophylaxis was done to remove stains caused by Chlorhexidine in the patients of control group.

### 2.3. Wound Healing Evaluation

### Measurement Procedure

Patients were monitored at 7 days after surgery. All patients were recalled and examined at 7 days and at one month for evaluation.

One week after surgery and after suture removal, the Early-wound healing-index (EHI) (measured using the method disclosed in Wachtel H et al. “Microsurgical access flap and enamel matrix derivative for the treatment of periodontal intra-bony defects: a controlled clinical study”, J Clin Periodontol. 2003, vol. 30, pp. 496-504) was assessed by one of the blinded specialists in five different degrees with a scale from 5 to 1:

1. Complete flap closure without fibrin line (5).

2. Complete flap closure with fibrin line (4).

3. Complete flap closure with small fibrin clot(s) (3).

4. Incomplete flap closure with partial necrosis (2).

5. Incomplete flap closure with complete necrosis (more than 50% of the former flap is involved) (1).

The wound area was classified as either partially or fully keratinized. In case of partial keratinization, the wound area was again classified as partially or fully keratinized 1 week later.

### 2.4. Postsurgical Pain or Discomfort Evaluation

### Measurement Procedure

The pain and discomfort were evaluated by the patient using a modified visual analogue scale (VAS) from 0 to 10, wherein 0 indicates no pain, 5 indicates moderate pain and 10 indicates maximum pain, according to their subjective feelings. The pain evaluation was performed at 2, 4 and 7 days of the intervention by telephone contact. The visual analogue scale (VAS) is a scale used to determine the pain intensity experienced by individuals.

The VAS consists of a line, approximately from 10-15 cm in length, with the left side signifying no pain with a smiling face image and the right side signifying the worst pain ever with a frowning face image. The VAS is used to assist individuals to determine pain levels, who may not be accustomed to rating their pain on other types of scales, such as a numeric rating scale.

### 2.5. Extraction Site Infection Evaluation

### Measurement Procedure

The assessment of extraction site infection was performed by the specialist. In case of infection, the specialist documented the type (i.e., alveolitis, osteitis, dental abscesses). The measurement of eating difficulties, swelling, and pain was done by daily reporting in a patient's diary through the Visual Analogue Scale (VAS) as disclosed above. These patients have been excluded from the study because of the difficulty of measuring.

### 2.6. Ulcer Heating Evaluation

The assessment of the ulcer area was performed by the specialist using the visual analogue scale (VAS) as disclosed in the present application. Patients with ulcer were instructed to rinse with 15 ml of the composition of the invention for 1 min twice daily after their regular homecare for 1 week. A visual evaluation of the ulcer was performed at the time pointed herein above.

### 2.7. Lesions Caused by Bacteria, Virus, or Fungi Evaluation

The assessment of the lesions caused by bacteria, virus or fungi was performed by the specialist using the visual analogue scale (VAS) as disclosed herein.

Patients with bacteria, virus or fungi were instructed to rinse with 15 ml of the composition of the invention for 1 min twice daily after their regular homecare for 1 week. A visual evaluation of the ulcer was performed at the time pointed herein above.

### 2.8. Results

The results obtained after performing the above-mentioned wound healing, ulcer heating, lesions caused by bacteria, virus or fungi and postsurgical pain or discomfort evaluation are summarized.

### Healing and Pain Evaluation

The results of the wound healing and pain/discomfort evaluation as well as the P-value are summarized in

TABLE 4

| | | | |
|---|---|---|---|
| Variable | | Comparative | |
| Evaluation | Example 2 | Example 1 | P-value |

Healing index

| | | | |
|---|---|---|---|
| Molars | 4 [4-4] | 3 [3-3] | <0.0001 |
| | n = 21 | n = 20 | |
| Central | 4.5 [4-5] | 3[3-3.75] | <0.0001 |
| | n = 16 | n = 17 | |
| Premolar | 5 [4-5] | 3 [3-3.5] | <0.0001 |
| | n = 19 | n = 18 | |
| All sites | 4 [4-5] | 3 [3-3] | <0.0001 |
| | n = 39 | n = 42 | |

Pain Score

| | | | | |
|---|---|---|---|---|
| Pain day 2 | 3 | [0-4] | 4 [0-7] | 0.1340 |
| Pain day 4 | 0 | [0-2] | 2 [0-5] | 0.0074 |
| Pain day 7 | 0 | [0] | 0 [0-3] | 0.0098 |

n represent the number of volunteers analysed in each case

### Ulcer Heating

After a visual evaluation of the ulcer mucosal area, it can be concluded that the compositions of the present invention allow achieving a complete healing of ulcers in a shorter period of time (7 days); meanwhile the commercial comparative composition requires more time to achieved the complete healing of the ulcered are of the oral mucosa. The disappearance of the pain was observed in 3 days after applying the solution of the invention according to Visual Analogue Scale (VAS).

### Mucosal Lesions Caused by Bacteria, Virus, or Fungi

Regarding the viral lesions caused by coronavirus (COVID-19), it was observed the healing of the lesions in a short period of time after complete healing, that is after 7 days; reducing at the same time the discomfort of the patients was observed in 4 days applying the solution of the invention according to Visual Analogue Scale.

### 3. Nasal Study Design

The study was made to patients with sinus pathologies such as sinusitis, this study was made in a split mouth, in one sinus was made the sinus wash with the composition of the present invention (Examples 1 or 2), and in the other side was made the sinus wash with physiological serum.

### 3.1. Population

Patients (female or male) who voluntarily signed the informed consent were enrolled to the study according to the following inclusion and exclusion criteria:

### Inclusion Criteria

Patient ≥14 years old and able to understand the nature of the proposed surgery and to sign the informed consent.
Patients systemically healthy.
Non-smokers patients.
No local or systemic antibiotic or antiseptic treatments in the previous 3 months.
No systemic diseases or conditions that could interfere with the study outcomes (pregnant or lactating females

### Exclusion Criteria

Smoker patients
Local or systemic conditions that would interfere with routine periodontal therapy.
Patients taking medications that cause gingival enlargement or the presence of gingival idiopathic overgrowth.
   Removal of Patients from Therapy or Assessment

Participants were free to withdraw from the study at any time, without any prejudice and without having to justify the reason. If the patient discontinued prematurely from the study, any relevant evaluations and observations and reasons for study discontinuation were recorded in the Case Report Form (CRF). The specialist might discontinue a participant from the study if there was a reasonable cause. In case of medical reason for withdrawal, the patient was to be monitored until complete recovery.

### 3.2. Treatments

### 3.2.1. Treatments Administered

Patients with sinus pathologies such as sinusitis, this study was made in a split mouth, in one sinus was made the sinus wash with the composition of the present invention (Examples 1 or 2), and in the other side was made the sinus wash with physiological serum.

### 3.2.2. Groups:

Control Group (CG): receiving comparative composition of physiological serum, and
Test Group (TG): receiving the spray composition of Example 3 of the invention.

### 3.2.3. Visit Schedule

The study was scheduled in two visits. The first visit was the surgery day (visit 1) and the second which was one week later the visit 1 (visit 2). Table 5 shows the assessments and indicates with an “X” the visits at which they were performed.

TABLE 5

| | Visit 1 | |
|---|---|---|
| | (baseline) | Visit 2 |
| | (surgery day) | (Week 1) |
| Informed consent | X | |
| Collection of clinical data | X | X |
| Nasal examination | X | X* |
| Inclusion/exclusion criteria | X | |
| Treatment | X | X |
| Patients dairy: clinical hilling measurements, | X | X* |
| eating difficulty and pain in a 0-10 scale (VAS) | | |
| Tolerability and post-treatment side effects | 0 | 0 |

*Exploratory endpoint.

### 3.2.4. Study Steps

All clinical measurements were performed by two specialists previously calibrated. All patients taking part in the study received a diagnostic workup including a clinical examination, photographs, and standardized periapical radiographs to evaluate the proposed surgical sites. Clinical registrations were made by the two trained and calibrated specialists.

### 3.3. Wound Healing Evaluation

Patients were monitored at 7 days after surgery. All patients were recalled and examined at 7 days One week after sinus cleaning with the invention solution, the wound healing was measured visually and by Xray.

### 3.4. Pain or Discomfort Evaluation

The pain and discomfort were evaluated by the patient using the modified visual analogue scale (VAS) defined herein from 0 to 10, wherein 0 indicates no pain, 5 indicates moderate pain and 10 indicates maximum pain, according to their subjective feelings. The pain evaluation was performed at 2, 4 and 7 days of the intervention by telephone contact.

### 3.5. Ulcer Heating Evaluation

Patients were monitored at 7 days after rinsing with the invention solution or with the comparative physiological serum. All patients were recalled and examined at 7 days. One week after, the wound healing of ulcer was measured visual

### 3.6. Lesions Caused by Bacteria, Virus, or Fungi Evaluation

Patients were monitored at 7 days after rinsing with the invention solution or with the comparative physiological serum. All patients were recalled and examined at 7 days. One week after, the wound healing of lesions caused by bacteria, virus or fungi was measured visual

### 3.7. Results

All patients treated with the solution of the current invention, having nasal/paranasal disease or conditions such as sinus injuries like sinusitis or oral lesions caused by bacteria, virus, or fungi, recovery, and regeneration of the epithelium of the nasal and paranasal tissue has been observed. It has also been observed and the disappearance of the symptoms of these pathologies such as pain.

### 4. Conclusion

The above disclosed experimental data regarding the use of the composition of the present invention as re-epithelization promoter of mucosa show that the compositions of the comprising a therapeutically effective amount of sodium dihydrogen phosphate and sodium chloride allows increasing and accelerating the re-epithelization and protection of the epithelium of the mucosa; as well as a reduction of pain and inflammation allowing an improvement of the general comfort of the patient during the process of mucosa healing. As a consequence of everything mentioned above, the compositions of the present invention reduce the time of treatment required for the healing of the mucosa. This fact, in combination with a reduction of patient discomfort results in a higher compliance of the treatment, and a low percentage of abandonment in comparison with the compositions disclosed in the state of the art.

For reasons of completeness, various aspects of the invention are set out in the following numbered clauses:

Clause 1. A water-based salt solution composition comprising:
a therapeutically effective amount of sodium dihydrogen phosphate;
a therapeutically effective amount of sodium chloride;
optionally, one or more pharmaceutically acceptable excipients of carriers; and
optionally, one or more additional active ingredients;
for promoting mucosal re-epithelialization.

Clause 2. The composition for use according to clause 1, wherein the mucosa is selected from the group consisting of oral mucosa, nasal mucosa, and paranasal mucosa.

Clause 3. The composition for use according to any of the clauses 1 or 2, wherein sodium dihydrogen phosphate is in a concentration from 8 to 12 mM in the solution and sodium chloride is in a concentration from 130 to 140 mM in the solution.

Clause 4. The composition for use according to any of the clauses 1-3, further comprising a therapeutically effective amount of potassium hydrogen phosphate and a therapeutically effective amount of potassium chloride.

Clause 5. The composition for use according to clause 4, wherein potassium hydrogen phosphate is in a concentration from 1.4 to 2.2 mM in the solution, and potassium chloride is in a concentration from 2.3 to 3.1 mM in the solution.

Clause 6. The composition for use according to any of the clauses 1-5, further comprising a therapeutically effective amount calcium salt and a therapeutically effective amount of salt of a divalent metal different from calcium.

Clause 7. The composition for use according to clause 6, wherein the calcium salt is calcium chloride and the salt of a divalent metal different from calcium is magnesium chloride.

Clause 8. The composition for use according to any of the clauses 6 or 7, wherein the calcium salt is in a concentration from 0.7 to 15 mM in the solution and the salt of a divalent metal different from calcium is in a concentration from 0.2 to 12 mM in the solution.

Clause 9. The composition for use according to any of the clauses 1-8, comprising:
a therapeutically effective amount of sodium dihydrogen phosphate;
a therapeutically effective amount of sodium chloride;
a therapeutically effective amount of potassium hydrogen phosphate;
a therapeutically effective amount of potassium chloride;
optionally, one or more pharmaceutically acceptable excipients of carriers; and
optionally, one or more additional active ingredients;
particularly,
sodium dihydrogen phosphate in a concentration from 8 to 12 mM in the solution;
sodium chloride in a concentration from 130 to 140 mM in the solution;
potassium hydrogen phosphate in a concentration from 1.4 to 2.2 mM in the solution;
potassium chloride in a concentration from 2.3 to 3.1 mM in the solution;
optionally, one or more pharmaceutically acceptable excipients of carriers; and
optionally, one or more additional active ingredients.

Clause 10. The composition for use according to any of the clauses 1-8, comprising:
a therapeutically effective amount of sodium dihydrogen phosphate;
a therapeutically effective amount of sodium chloride;
a therapeutically effective amount of potassium hydrogen phosphate;
a therapeutically effective amount of potassium chloride;
a therapeutically effective amount of calcium chloride;
a therapeutically effective amount of a salt of a divalent metal different from calcium, particularly magnesium chloride;
optionally, one or more pharmaceutically acceptable excipients of carriers; and optionally, one or more additional active ingredients;
particularly,
sodium dihydrogen phosphate in a concentration from 8 to 12 mM in the solution;
sodium chloride in a concentration from 130 to 140 mM in the solution;
potassium hydrogen phosphate in a concentration from 1.4 to 2.2 mM in the solution;
potassium chloride in a concentration from 2.3 to 3.1 mM in the solution;
calcium chloride in a concentration from 0.7 to 15 mM in the solution;
a salt of a divalent metal different from calcium, particularly magnesium chloride, in a concentration from 0.2 to 12 mM in the solution;
optionally, one or more pharmaceutically acceptable excipients of carriers; and
optionally, one or more additional active ingredients.

Clause 11. The composition for use according to any of the clauses 1-10, wherein the pH is from 6.8 and 7.4; particularly about 7.

Clause 12. The composition for use according to any of the clauses 1-11, wherein the mucosal re-epithelialization comprises wound healing; ulcer healing; healing of lesions caused by bacteria, virus, and fungi; healing of bacterial, viral, or fungal infection and after any oral or maxillofacial surgery.

Clause 13. The composition for use according to any of the clauses 1-12, in the treatment of a mucosal disease or condition which involves the re-epithelialization of the mucosa; particularly in the treatment of the gingival disease or condition.

Clause 14. The composition for use according to any of the clauses 1-13, which comprises contacting once or twice a day the mucosa with the composition as defined in any of the clauses 1-11 for a period of 4 to 10 days.

Clause 15. A composition comprising:
sodium dihydrogen phosphate in a concentration from 8 to 12 mM in the solution;
sodium chloride in a concentration from 130 to 140 mM in the solution;
potassium hydrogen phosphate in a concentration from 1.4 to 2.2 mM in the solution;
potassium chloride in a concentration from 2.3 to 3.1 mM in the solution;
calcium salt, particularly calcium chloride, in a concentration from 0.7 to 2 mM in the solution;
a salt of a divalent metal different from calcium, particularly magnesium chloride, in a concentration from higher than 0.2 to 1.5 mM in the solution;
optionally, one or more pharmaceutically acceptable excipients of carriers; and
optionally, one or more additional active ingredients.

Clause 16. A process for re-epithelization which comprises:
a) directly applying the composition as defined in any of the clauses 1-11 and 15 to the area to be treated; or alternatively,
b) applying the compositions as defined in any of the clauses 1-11 and 15 to suitable means for the subsequent application to those means to the area to be treated.

### CITATION LIST

1. O'Leary T J et al. “The plaque control record”. J. Periodontol. 1972, vol 43(1), pp. 38.
2. Ainamo J et al. “Problems and proposals for recording gingivitis and plaque”. I. Int. Dent. J., 1975, vol. 25(4), pp. 229-35.
3. Wachtel H et al. “Microsurgical access flap and enamel matrix derivative for the treatment of periodontal intra-bony defects: a controlled clinical study”, J Clin Periodontol. 2003, vol. 30, pp. 496-504)

What is claimed is:

1. A treatment method, the method comprising administering to said mammal suffering from or susceptible to suffer from a musical disease or condition, said disease or condition implying a loss of integrity of an epithelium, an effective amount of a water-based salt solution composition comprising:
a therapeutically effective amount of sodium dihydrogen phosphate;
a therapeutically effective amount of sodium chloride;
optionally, one or more pharmaceutically acceptable excipients of carriers; and
optionally, one or more additional active ingredients.

2. The method according to claim 1, wherein the epithelium is of a mucosal cavity selected from the group consisting of oral, nasal, and paranasal mucosal cavity.

3. The method according to claim 1, wherein the sodium dihydrogen phosphate is in a concentration from 8 to 140 mM in the solution and the sodium chloride is in a concentration from 8 to 140 mM in the solution.

4. The method according to claim 1, wherein:
the sodium dihydrogen phosphate is in a concentration from 8 to 12 mM in the solution and the sodium chloride is in a concentration from 130 to 140 mM in the solution; or alternatively,
the sodium dihydrogen phosphate is in a concentration from 130 to 140 mM in the solution and the sodium chloride is in a concentration from 8 to 12 mM in the solution.

5. The method according to claim 1, wherein the water-based salt solution composition further comprises a therapeutically effective amount of potassium hydrogen phosphate and a therapeutically effective amount of potassium chloride.

6. The method according to claim 5, wherein the potassium hydrogen phosphate is in a concentration from 1.4 to 2.2 mM in the solution, and the potassium chloride is in a concentration from 2.3 to 3.1 mM in the solution.

7. The method according to claim 1 wherein the water-based salt solution composition further comprises a therapeutically effective amount a calcium salt and a therapeutically effective amount of a salt of a divalent metal different from calcium.

8. The method according to claim 7, wherein the calcium salt is calcium chloride and the salt of a divalent metal different from calcium is magnesium chloride.

9. The method according to claim 7, wherein the calcium salt is in a concentration from 0.7 to 15 mM in the solution and the salt of a divalent metal different from calcium is in a concentration from 0.2 to 12 mM in the solution.

10. The method according to claim 1, wherein the water-based salt solution composition further comprises:
a therapeutically effective amount of potassium hydrogen phosphate;
a therapeutically effective amount of potassium chloride;
optionally, one or more pharmaceutically acceptable excipients of carriers; and
optionally, one or more additional active ingredients;
particularly,
sodium dihydrogen phosphate in a concentration from 8 to 12 mM in the solution;
sodium chloride in a concentration from 130 to 140 mM in the solution;
potassium hydrogen phosphate in a concentration from 1.4 to 2.2 mM in the solution;
potassium chloride in a concentration from 2.3 to 3.1 mM in the solution;
optionally, one or more pharmaceutically acceptable excipients of carriers; and
optionally, one or more additional active ingredients.

11. The method according to claim 1, wherein the water-based salt solution composition further comprises:
a therapeutically effective amount of potassium hydrogen phosphate;
a therapeutically effective amount of potassium chloride;
a therapeutically effective amount of calcium chloride;
a therapeutically effective amount of a salt of a divalent metal different from calcium, particularly magnesium chloride;
optionally, one or more pharmaceutically acceptable excipients of carriers; and
optionally, one or more additional active ingredients;
particularly,
sodium dihydrogen phosphate in a concentration from 8 to 12 mM in the solution;
sodium chloride in a concentration from 130 to 140 mM in the solution;
potassium hydrogen phosphate in a concentration from 1.4 to 2.2 mM in the solution;
potassium chloride in a concentration from 2.3 to 3.1 mM in the solution;
calcium chloride in a concentration from 0.7 to 15 mM in the solution;
a salt of a divalent metal different from calcium, particularly magnesium chloride, in a concentration from 0.2 to 12 mM in the solution;
optionally, one or more pharmaceutically acceptable excipients of carriers; and
optionally, one or more additional active ingredients.

12. The method according to claim 1, wherein the pH of the composition is from 6.8 and 7.4; particularly about 7.

13. The method according to claim 1, wherein the administration of the composition promotes mucosal re-epithelialization and mucosal re-epithelialization comprises wound healing, ulcer healing, healing of lesions caused by bacteria, virus, and fungi, healing of bacterial, viral, or fungal infection and after any oral or maxillofacial surgery.

14. The method according to claim 1, wherein the treatment of the mucosal disease or condition involves the re-epithelialization of the mucosa, particularly the treatment of the gingival disease or condition.

15. The method according to claim 1, which comprises contacting once or twice a day the mucosa with the composition for a period of 4 to 10 days.

16. A water-based salt solution composition comprising:
sodium dihydrogen phosphate in a concentration from 8 to 12 mM in the solution;
sodium chloride in a concentration from 130 to 140 mM in the solution;
potassium hydrogen phosphate in a concentration from 1.4 to 2.2 mM in the solution;
potassium chloride in a concentration from 2.3 to 3.1 mM in the solution;
calcium salt, particularly calcium chloride, in a concentration from 0.7 to 2 mM in the solution;
a salt of a divalent metal different from calcium, particularly magnesium chloride, in a concentration from higher than 0.2 to 1.5 mM in the solution;
optionally, one or more pharmaceutically acceptable excipients of carriers; and
optionally, one or more additional active ingredients.
